SEQUENCE LISTING (20 sequences)

SEQ ID NO: 1 - DNA, 1518 bp, synthetic construct
  source 1..1518: mol_type = other DNA
atggttgactctaacagtacccttttgtactcagtcttctccgttgtttccatcgttattgccattaagttctggttgaactctcgtaggatcagaaacttgcctccttctccaccaagcatcccaattattggtcacttacatttgttgaagccaccagttcacagatctttgcaagctttgtcccaaaagtatggtccaatcttctctttgaaattcggttccagattggttgtggtcgttacctctatggaaattgctgaagaatgtttcaccaagaacgatattgtttttgctaacagaccagattttgctgtctccaactgtttgtcctacaacaacactaccatcggtgctgctccatacggtgaccactggagaaagttgagaagagtctctgccctcgaaatcttgtcttctaatagattgcaaatgaacttgggtatcagaagagacgaaattcgtcgtttggtcaagaaactatcttctcacggtgacttcgctaaggtcaacttgaagtctatcgtcaaagaactgaccttaaacatcacaatgagaatggttgctggtaagcggtattacggtgaagaagaaaaggaagacgatgaagccagaactttccaagttttggtcgatgaattatttgaattcactttatcctcctacccaggtgatttcttgccaattttgaagtacatcgacatccaaggtttcatgaaaagagctagaaaattgatgaagaaggttgatgctttctggcaaggtttaattgacgaacaccgtacttacaagaacggtaacgttgaaatggaaaactccatgatttctcatttgttgaagttgcaagaatctcaaccagaatactacactgaagatattatcaagggtttgatcttgaccatgattttgggtggttccgataccactgctgttactatagaatgggctatgtctaatctgttgaaccacccatctgttatgaacaaggctagagccgaattggaatctcaaatcgaaactgacaagctagtcgaagaatccgacttgtctaagttgtcttacttgagatgtatcatcttagaaaccttgagattatacccagctggtccattgttgttgccacacagatcctcagctgactccactttgtccaagtacaacgtcccatctgacactatcttgttggttaacgcttgggccattcacagagatccaaccttgtgggatgacccaacttctttccgtccagaaagattcgaaaatagtgaaggtgaagctcacaagttcgtcatctctttcggtttgggcagaagagcttgcccaggtactggtatggcccatagagttttaggtttgacactaggtaccttcatccaatgtttcgaatgggacagaattgaaggtaaggaaattgacatggctgaaggtaagggtgtttccatgccaaaggctgagccattggaagcattgtgtaagccaagacacgtcgctatcaaggccatttcctcaaccttttga

SEQ ID NO: 2 - DNA, 840 bp, synthetic construct
  source 1..840: mol_type = other DNA
ctattgacccaaggttttggcagtgaattcgaaagatggattagtcaaagagaaaccaccatcaacaaccaagttcaaaccagacacgtatttggcttcatcagaacccaaataaacagcagctttagcaacatcttcagcttccatagcagcacctttcaaattaccggacatggagatgacgtattccatcatctttttctcttcctcgttcatagatggcaacaacattctagtaacggtagcgaatggagaaatgcagttgactctaataccatgtttacccaattcaacagccaaggtcttagtcataccaactatagcatgttttgtagcagagtaagctggggtgttaccagaacaaataacagattccatagaagaggtgaacaggatacaacctttcttagctggaatcataactctggcagcatgtttagcacccataaaagtaccgaaaacattgacgtcgaaaacgtgtctgaattccttcatatcagtggtttcagtcttagaagtgacgttaccacaaataccagcgttgttgaacatgatgtccaaagtaccatacttgtcaatggcgaagttaacagcgttttcaatatcggtttcgtcagtaacatcacaatgcaagtaggagatctcacgaccaatagaacccaattctttacacaaggtgtcagccaaatcatctctaacatcagcgataataaccttggcaccttctctagtgaataatctaacagtagattcacccaaaccagaagcaccaccagtaatcaaagcaaccttaccatccaatctttttggaatggatggagattgaccattagatgacat

SEQ ID NO: 3 - DNA, 30 bp, synthetic construct
  source 1..30: mol_type = other DNA
tttgccaacaatcgaaaccaaacatatatc

SEQ ID NO: 4 - DNA, 53 bp, synthetic construct
  source 1..53: mol_type = other DNA
caaatcttaaagtcatacattgcacgactaatataaagcagccgctaccaaac

SEQ ID NO: 5 - DNA, 53 bp, synthetic construct
  source 1..53: mol_type = other DNA
gtttggtagcggctgctttatattagtcgtgcaatgtatgactttaagatttg

SEQ ID NO: 6 - DNA, 59 bp, synthetic construct
  source 1..59: mol_type = other DNA
ggtactgttagagtcaaccatttattgtaatatgtgtgtttgtttggattattaagaag

SEQ ID NO: 7 - DNA, 53 bp, synthetic construct
  source 1..53: mol_type = other DNA
ccaaacaaacacacatattacaataaatggttgactctaacagtacccttttg

SEQ ID NO: 8 - DNA, 50 bp, synthetic construct
  source 1..50: mol_type = other DNA
ggtagattgggctacgtaaattcgatcaaaaggttgaggaaatggccttg

SEQ ID NO: 9 - DNA, 50 bp, synthetic construct
  source 1..50: mol_type = other DNA
aggccatttcctcaaccttttgatcgaatttacgtagcccaatctaccac

SEQ ID NO: 10 - DNA, 59 bp, synthetic construct
  source 1..59: mol_type = other DNA
gcataaaaacttcatgaaaattcggcagaaaataagcggcgccacttctataaaaggtc

SEQ ID NO: 11 - DNA, 59 bp, synthetic construct
  source 1..59: mol_type = other DNA
actttaaaatttgtatacacttattttttttataactttaggatttaatgcaggtgacg

SEQ ID NO: 12 - DNA, 59 bp, synthetic construct
  source 1..59: mol_type = other DNA
atctttcgaattcactgccaaaaccttgggtcaataggatctcccatgtctctactggt

SEQ ID NO: 13 - DNA, 59 bp, synthetic construct
  source 1..59: mol_type = other DNA
aaagaagcaccaccaccagtagagacatgggagatcctattgacccaaggttttggcag

SEQ ID NO: 14 - DNA, 50 bp, synthetic construct
  source 1..50: mol_type = other DNA
ctaatctaagttttaattacaaaatgtcatctaatggtcaatctccatcc

SEQ ID NO: 15 - DNA, 57 bp, synthetic construct
  source 1..57: mol_type = other DNA
gattgaccattagatgacattttgtaattaaaacttagattagattgctatgctttc

SEQ ID NO: 16 - DNA, 50 bp, synthetic construct
  source 1..50: mol_type = other DNA
cttacgatacctgagtattcccacagttagtgatcccccacacaccatag

SEQ ID NO: 17 - DNA, 50 bp, synthetic construct
  source 1..50: mol_type = other DNA
gaagctatggtgtgtgggggatcactaactgtgggaatactcaggtatcg

SEQ ID NO: 18 - DNA, 50 bp, synthetic construct
  source 1..50: mol_type = other DNA
atataaaggcgcctggccgattaagcaaggattttcttaacttcttcggc

SEQ ID NO: 19 - DNA, 50 bp, synthetic construct
  source 1..50: mol_type = other DNA
cgaagaagttaagaaaatccttgcttaatcggccaggcgcctttatatca

SEQ ID NO: 20 - DNA, 44 bp, synthetic construct
  source 1..44: mol_type = other DNA
gactataatattatgcatataggatataccaaaaattctctctg

(19) United States
(12) Patent Application Publication
(10) Pub. No.: US 2026/0193620 A1
(43) Pub. Date: Jul. 9, 2026

(54) HcCYP81Q58 TRANSGENIC SACCHAROMYCES CEREVISIAE ENGINEERING BACTERIA OF HEMSLEYA CHINWNSIS CYTOCHROME OXIDASE AND APPLICATION THEREOF

(71) Applicants: Yunnan Agricultural University, Kunming City (CN); Yunnan Yunke Characteristic Plant Extraction Laboratory Co., Ltd., Kunming City (CN)

(72) Inventors: Guanghui ZHANG, Kunming City (CN); Zhiyuan LI, Kunming City (CN); Shengchao YANG, Kunming City (CN); Bing HAO, Kunming City (CN); Geng CHEN, Kunming City (CN); Yanyu SHU, Kunming City (CN); Xiaolin FENG, Kunming City (CN); Dina MA, Kunming City (CN); Simei HE, Kunming City (CN); Yingchun LU, Kunming City (CN)

(73) Assignees: Yunnan Agricultural University, Kunming City (CN); Yunnan Yunke Characteristic Plant Extraction Laboratory Co., Ltd., Kunming City (CN)

(21) Appl. No.: 19/012,943

(22) Filed: Jan. 8, 2025

(51) Int. Cl. C12N 9/02; C12N 15/81; C12P 5/00

(52) U.S. Cl. CPC C12N 9/0071; C12N 15/81; C12P 5/007

(57) ABSTRACT

The invention discloses a HcCYP81Q58 gene of Hemsleya chinensis cytochrome oxidase, a sequence of the gene is shown in SEQ NO:1. The invention constructs a genetically modified Saccharomyces cerevisiae engineering bacteria of Cuol-04-1, it is capable of producing a variety of cucurbitacin intermediates at a high level, including 11-Carbonyl-cucurbita-5,23-diene-3β, 20,25-triol, 11-Carbonyl-cucurbita-5,24-diene-3β, 20,23-triol, 11-Carbonyl-cucurbita-5,23-diene-20,25-diol-3β-one, and 11-Carbonyl-cucurbita-5,24-diene-20,23-diol-3β-one, it provides a source for the production of cucurbitacins.

This instant application contains a Sequence Listing which has been submitted electronically in XML file format and is hereby incorporated by reference in its entirety. Said XML Copy, created on Jan. 8, 2025, is named Sequence table.xml and is 22000 bytes in size.

### TECHNICAL FIELD

The invention belongs to the field of biotechnology and specifically relates to an application of a HcCYP81Q58 of Hemsleya chinensis cytochrome oxidase and its transgenic engineering bacteria in a preparation of cucurbitacin intermediates.

### BACKGROUND OF THE INVENTION

Cucurbitacins are highly oxidized tetracyclic triterpenoids with high medicinal value, especially in anti-tumor. Due to the toxic and side effects of cucurbitacin, its clinical application is limited. Therefore, only a small number of cucurbitacins have been developed as drugs (CuB, dihydrocucurbitacin I, etc.). Cucurbitacins are rich in the tubers of Hemsleya chinensis plants. In particular, dihydrocucurbitacin (a mixture of dihydrocucurbitacin I and dihydrocucurbitacin II) extracted from the tubers of Hemsleya chinensis plants is a raw material for the production of Chinese medicine dihydrocucurbitacin tablets (protected varieties of traditional Chinese medicine) and dihydrocucurbitacin capsules, the drug has the functions of clearing heat and detoxifying, anti-bacterial and anti-inflammatory. It is mainly used in the treatment of bacillary dysentery, enteritis, bronchitis, acute tonsillitis, and other diseases. Unlike other cucurbitacins, the researchers found that cucurbitacin Ia (dihydrocucurbitacin I) has a significant cytotoxic effect to inhibit the proliferation of tumor cells by inhibiting JAK2/STAT3 downstream of survivin (Survivin), making it a new anticancer drug.

Cytochrome oxidase CYP450 is a heme-iron-sulfur protein encoded by a supergene family. In eukaryotes, CYP450 oxidase is a membrane-anchored protein that binds to organelles such as endoplasmic reticulum, mitochondria, plastids, and Golgi apparatus. It is mainly located in the endoplasmic reticulum and participates in the metabolism and oxidation of a variety of important substances in organisms. It can insert an oxygen atom into a hydrophobic molecule in a biochemical reaction to obtain higher activity or hydrophilicity, also known as mixed-function oxidase (MFO).

Studies have shown that cucurbitacin biosynthesis starts from the cyclization of 2,3-oxidosqualene to form cucurbitadienol (Cuol), which is catalyzed by cucurbitadienol synthase (CBS) of the oxidized squalene cyclase (OSCs) family. Cucurbitadienol is catalyzed by cytochrome P450 by introducing hydroxyl, carcassetteyl, or epoxy groups for oxidative modification of specific sites, resulting in a variety of tetracyclic triterpenoids. CYP69 can catalyze the C24,25 hydroxylation reaction of cucurbitadienol, and the hydroxyl configuration at C24 has two configurations of a and 3.

Cucurbitacins are highly oxidized and have low content in plants, so the extraction and purification process is complex and time-consuming, and a lot of manpower and material resources are needed. At present, the development of related drugs is mainly prepared by extraction from plants. Nowadays, there are still many challenges in the chemical synthesis of cucurbitacins. Especially the key intermediate compounds of cucurbitacin. The development of synthetic biology and metabolic engineering provides a new opportunity for the efficient heterologous synthesis of tetracyclic triterpenoids. As a eukaryotic expression system, Saccharomyces cerevisiae has a clear genetic background and a mature genetic modification strategy, the endogenous mevalonate (MVA) pathway can provide the required precursors for the synthesis of more terpenoids, which is conducive to the synthesis of terpenoids. Therefore, in the study of the cucurbitacin biosynthesis pathway and the possible production in the future, it is very important to build and improve Saccharomyces cerevisiae engineering bacteria with the efficient production of cucurbitacin intermediates 11-Carbonyl-cucurbita-5,23-diene-3β, 20,25-triol; 11-Carbonyl-cucurbita-5,24-diene-3β, 20,23-triol; 11-Carbonyl-cucurbita-5, 23-diene-20, 25-diol-3β-one and 11-Carbonyl-cucurbita-5, 24-diene-20, 23-diol-3β-one.

### SUMMARY OF THE INVENTION

In view of the shortcomings of the existing technology, the purpose of the invention is to provide a HcCYP81Q58 of Hemsleya chinensis cytochrome oxidase, the enzyme can produce cucurbitacin intermediates at a high level.

To realize the invention, the invention adopts the following technical solution.

In a first aspect, the invention discloses a HcCYP81Q58 gene of Hemsleya chinensis cytochrome oxidase, and a sequence of the gene is shown in SEQ NO:1.

Preferably, an application of the Hemsleya chinensis cytochrome oxidase in a preparation of cucurbitacin intermediates.

Preferably, the cucurbitacin intermediates comprising:
11-Carbonyl-cucurbita-5, 23-diene-3β, 20, 25-triol (5),
11-Carbonyl-cucurbita-5, 24-diene-3β, 20, 23-triol (5b),
11-Carbonyl-cucurbita-5, 23-diene-20, 25-diol-3β-one (5a) and
11-Carbonyl-cucurbita-5, 24-diene-20, 23-diol-3β-one (5c).

In a second aspect, the invention discloses an expression vector, it is a gene expression cassette composed of a promoter and HcCYP81Q58, HcSDR34, LEU2 genes, and a terminator in series.

Preferably, the promoter is composed of GPMp, and TEF1p promoters in series; the terminator is composed of IDP1t, PGK1t in series.

In a third aspect, the invention discloses a method for constructing a HcCYP81Q58 transgenic Saccharomyces cerevisiae engineering bacteria of Hemsleya chinensis cytochrome oxidase, comprising the following steps:

controlling an expression of HcCYP81Q58 gene by using a constitutive promoter GPMp and a constitutive terminator IDP1t; controlling an expression of HcSDR34 gene by using a constitutive promoter TEF1p and a constitutive terminator PGK1t, and using LEU2 as a screening tag; constructing gene expression cassettes, the gene expression cassettes become downstream modules of a synthesis pathway of cucurbitacin intermediates; inserting gene expression cassettes into a γDNA site of a Saccharomyces cerevisiae by co-transformation, and carrying out an assembly by Saccharomyces cerevisiae homologous recombination ability to form Saccharomyces cerevisiae engineering bacteria containing a complete biosynthesis pathway of cucurbitacin intermediates.

Preferably, the constitutive promoter is a combination of promoters GPMp, and TEF1p.

Preferably, the constitutive terminator is a combination of terminators IDP1t, PGK1t.

Preferably, the gene expression cassettes also comprise a LEU2 gene, the LEU2 gene is a screening gene.

Preferably, the cucurbitacin intermediates comprise:
11-Carbonyl-cucurbita-5,23-diene-3β, 20,25-triol (5),
11-Carbonyl-cucurbita-5, 24-diene-3β, 20, 23-triol (5b),
11-Carbonyl-cucurbita-5,23-diene-20,25-diol-3β-one (5a) and
11-Carbonyl-cucurbita-5,24-diene-20,23-diol-3β-one (5c).

The beneficial effect of the invention is that the gene HcCYP81Q58 is successfully excavated through the genome and transcriptome analysis of the traditional Chinese medicine Hemsleya chinensis. It is found that the enzyme can catalyze the C25 hydroxylation of 11-carbonyl-20β-hydroxy-Cuol to form 11-Carbonyl-cucurbita-5,23-diene-3β, 20,25-triol, which can then be catalyzed by HcSDR34 to convert C3-ketone to 11-Carbonyl-cucurbita-5. 23-diene-20,25-diol-3β-one; at the same time, HcCYP81Q58 can also catalyze the C23 hydroxylation of 11-carbonyl-20β-hydroxy-Cuol to form 11-Carbonyl-cucurbita-5,24-diene-3β, 20,23-triol, which can be converted to 11-Carbonyl-cucurbita-5,24-diene-20,23-diol-3β-one by C3-ketone catalyzed by HcSDR34 to form multiple cucurbitin intermediates.

### BRIEF DESCRIPTION OF DRAWINGS

The figures are used to provide further understanding of the invention and form part of the specification, the figures are used to explain the invention together with the embodiment of the invention and do not constitute a restriction on the invention. In the attached figures:

FIG. 1 is a construction of Saccharomyces cerevisiae engineering bacteria Cuol-04-1.

FIG. 2 is a schematic diagram of the synthetic pathway of the HcCYP81Q58 (where OSC6 represents cucurbitadienol synthase from Hemsleya chinensis; HcCYP87D20 represents the cucurbit β-hydroxylase at the C20 position of cucurbit enol C11 ketone derived from Hemsleya chinensis plants);

FIG. 3A is Extracted ion chromatograms (EICs) of product 5, 5a, 5b and 5c, EICs are displayed for m/z of 495 (calculated mass for (5) [M]+), 493 (calculated mass for (5a) [M]+), 495 (calculated mass for (5b) [M]+) and 493 (calculated mass for (5c) [M]+); FIG. 3B is mass spectra of product 5, 5a and FIG. 3C is mass spectra of product 5b, 5c;

FIGS. 4A-4B are an NMR spectrum of product 5 of HcCYP81Q58 gene (FIG. 4A is an ¹³C NMR spectrum in CDCl₃ at 600 MHz of product 5; FIG. 4B is an ¹H NMR spectrum in CDCl₃ at 600 MHz of product 5; Detailed information is provided in Table 3);

FIGS. 5A-5B are the NMR spectrum of product 5a of HcCYP81Q58 gene (FIG. 5A is an ¹³C NMR spectrum in CDCl₃ at 600 MHz of product 5a; FIG. 5B is an ¹H NMR spectrum in CDCl₃ at 600 MHz of product 5a; Detailed information is provided in Table 3);

FIGS. 6A-6B are an NMR spectrum of product 5b of HcCYP81Q58 (FIG. 6A is an ¹³C NMR spectrum in CDCl₃ at 600 MHz of product 5b; FIG. 6B is an ¹H NMR spectrum in CDCl₃ at 600 MHz of product 5b; Detailed information is provided in Table 3);

FIGS. 7A-7B are an NMR spectrum of product 5c of HcCYP81Q58 (FIG. 7A is an ¹³C NMR spectrum in CDCl₃ at 600 MHz of product 5c; FIG. 7B is an ¹H NMR spectrum in CDCl₃ at 600 MHz of product 5c; Detailed information is provided in Table 3);

### DETAILED DESCRIPTION OF THE INVENTION

The following will explain the scheme of the invention in combination with an embodiment. Technicians in this field will understand that the following embodiment is only used to illustrate the invention and should not be regarded as a limitation to the scope of the invention. If the specific technology or conditions are not specified in the embodiment, it shall be carried out in accordance with the technology or conditions described in the literature in this field or by the product description. Reagents or instruments used that do not specify the manufacturer are conventional products that can be obtained through commercial purchase.

Embodiment 1: This embodiment provides a method for constructing recombinant Saccharomyces cerevisiae Coul-04-1.

### 1. Optimizing and Cloning the HcCYP81Q58 Gene:

(1) HcCYP81Q58 with Saccharomyces cerevisiae codon optimization: The HcCYP81Q58 is transferred to Wuhan Jinkairui Biological Engineering Co., Ltd. for gene synthesis and codon optimization;
(2) Optimized HcCYP81Q58 amplification and recovery: The HcCYP81Q58 is synthesized and optimized by Wuhan Jinkairui Bioengineering Co., Ltd. in the Y33 plasmid, and the gene is amplified by PCR, and then electrophoresis is carried out, after confirming the success of the amplification, the target band is recovered, the HcCYP81Q58 is identified as shown in SEQ NO: 1;
PCR reaction procedure: 95° C., 3 min; 95° C., 15S, 58° C., 45S, 72° C., 1 min, 35 cycles; 72° C., 10 min.

### 2. The Activation and Culture Method for the Transgenic Engineering Host Bacteria Comprises the Following Steps:

(1) The host bacteria of Saccharomyces cerevisiae is transferred to YPD liquid medium for activation, and the Saccharomyces cerevisiae liquid is obtained, the activation condition is 30° C., 220 rpm for 24 h;
(2) The above-mentioned Saccharomyces cerevisiae liquid is transferred to YPD liquid medium for culture, and the Saccharomyces cerevisiae is collected;
(3) The Saccharomyces cerevisiae cells are resuspended in deionized water and centrifuged to obtain Saccharomyces cerevisiae precipitation, the culture method is as follows: the initial OD600 is 0.4, it is cultured at 30° C. and 220 rpm until the OD600 reached 0.8-0.9.
   3. The Method for Constructing Recombinant Saccharomyces cerevisiae Coul-04-1 Comprises the Following Steps:
(1) Constructing Saccharomyces cerevisiae engineering bacteria Cuol-04-1 that can product 11-Carbonyl-cucurbita-5,23-diene-3β, 20,25-triol, 11-Carbonyl-cucurbita-5,24-diene-3β, 20,23-triol, 11-Carbonyl-cucurbita-5,23-diene-20,25-diol-3β-one, 11-Carbonyl-cucurbita-5,24-diene-20,23-diol-3β-one at a high level:

The genes ERG20, ERG9, ERGI and tHMG1 related to mevalonate pathway from Saccharomyces cerevisiae BY4742, the genes HcOSC6, HcCYP87D20 encoding cucurbitadienol synthase, and the gene HcCPR1 encoding cytochrome P450 reductase from Hemsleya chinensis plant are constructed into gene expression cassettes, which are used as modules for the production of 11-carbonyl-20β-hydroxy-Cuol (cucurbitadienol) synthesis pathway. On this basis, the constitutive promoter GPMp and the constitutive terminator IDP1t are used to control the expression ofHcCYP81Q58, and the constitutive promoter TEF1p and the constitutive terminator PGK1t are used to control the expression of HcSDR34 (HcSDR34 sequence is shown in SEQ NO: 2). Using LEU2 as the screening tag, the gene expression cassettes are constructed to become the downstream modules of the cucurbitacin intermediate synthesis pathway. The gene expression cassettes are inserted into the γDNA site in Saccharomyces cerevisiae by co-transformation. The Saccharomyces cerevisiae engineering bacteria Cuol-04-1 is constructed by overexpressing the key gene HcCYP81Q58 in the cucurbitacin pathway, the HcCYP81Q58 provides hydroxylation at C25 and C23 of cucurbitacin in Saccharomyces cerevisiae, and the HcSDR34 provides a enzyme-catalyzed reaction of carbonylation at C3 of cucurbitacin in Saccharomyces cerevisiae.

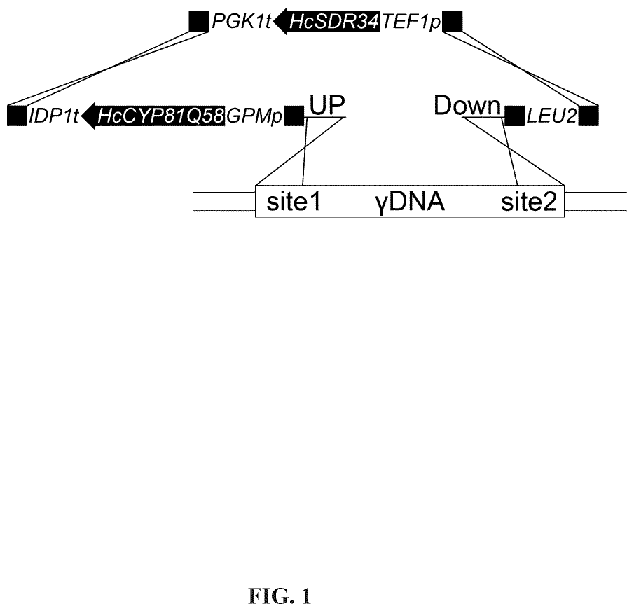

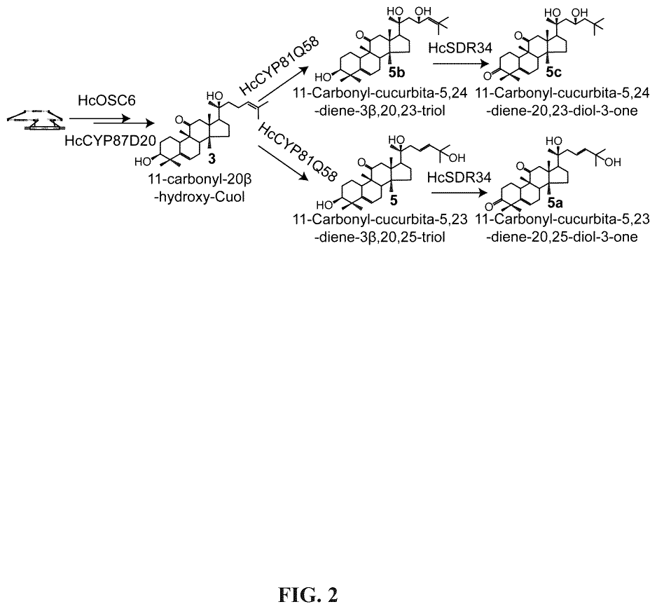

(2) The gene expression cassettes are co-transformed into the yDNA site of Saccharomyces cerevisiae Cuol-04-1 by lithium acetate transformation method, the Saccharomyces cerevisiae homologous recombination ability is used to assemble and contain the screening marker gene LEU2 to form a Saccharomyces cerevisiae engineering bacteria Cuol-04-1 in biosynthetic pathway with complete 11-Carbonyl-cucurbita-5,23-diene-3β, 20,25-triol (5), 11-Carbonyl-cucurbita-5,24-diene-3β, 20,23-triol (5b), 11-Carbonyl-cucurbita-5,23-diene-20,25-diol-3β-one (5a), 11-Carbonyl-cucurbita-5, 24-diene-20, 23-diol-3β-one (5c);
among them, the construction method for gene expression cassettes provided by the Saccharomyces cerevisiae engineering bacteria Cuol-04-1 is to use the constitutive promoter GPMp, and terminator IDPlt to control the expression of HcCYP81Q58 gene and form HcCYP81Q58 gene expression cassettes: UP-GPMp-HcCYP81Q58-IDP1t; the expression of HcSDR34 is controlled by promoter TEF1p and terminator PGK1t, and the HcSDR34 gene expression cassette is composed of TEF1p-HcSDR34-IPGK1t; and Using LEU2 as a screening tag, the gene expression cassettes were constructed to form a downstream module of the synthetic pathway for the production of cucurbitacin intermediates, and co-transformation was applied to insert the cassettes into γDNA sites in Saccharomyces cerevisiae. (FIG. 1).

The modules in the pathways of
11-Carbonyl-cucurbita-5,23-diene-3β, 20,25-triol (5),
11-Carbonyl-cucurbita-5,24-diene-3β, 20,23-triol (5b),
11-Carbonyl-cucurbita-5,23-diene-20,25-diol-3β-one (5a),
11-Carbonyl-cucurbita-5,24-diene-20,23-diol-3β-one (5c) are produced in recombinant engineering bacteria by the above gene expression cassettes, LEU2 is a screening marker. The construction method for gene expression cassettes is shown in FIG. 1.

Among them, the Saccharomyces cerevisiae transformation system comprises the following components: PEG4000 solution, LiAc solution, sSDNA, recombinant plasmid, and sterile deionized water. The standing time is 20 min, and the heat shock condition is maintained at 42° C. for 40 min.

TABLE 1
Primers used in the construction of engineering bacteria Cuol-04-1

| Primer | Sequence(5′to3′) |
|---|---|
| 1-UP-F | TTTGCCAACAATCGAAACCAAACATATATC |
| 1-UP-R | CAAATCTTAAAGTCATACATTGCACGACTAATATAAAGCAGCCG |
| | CTACCAAAC |
| 1-GPMP-F | GTTTGGTAGCGGCTGCTTTATATTAGTCGTGCAATGTATGACTTT |
| | AAGATTTG |
| 1-GPMP-R | GGTACTGTTAGAGTCAACCATTTATTGTAATATGTGTGTTTGTTT |
| | GGATTATTAAGAAG |
| 1-Hc81Q58- | CCAAACAAACACACATATTACAATAAATGGTTGACTCTAACAGT |
| F | ACCCTTTTG |
| 1-Hc81Q58- | GGTAGATTGGGCTACGTAAATTCGATCAAAAGGTTGAGGAAATG |
| R | GCCTTG |
| 1-IDP1t-F | AGGCCATTTCCTCAACCTTTGATCGAATTTACGTAGCCCAATCTA |
| | CCAC |
| 1-IDP1-R | GCATAAAAACTTCATGAAAATTCGGCAGAAAATAAGCGGCGCC |
| | ACTTCTATAAAAGGTC |
| 2-PGK1t-F | ACTTTAAAATTTGTATACACTTATTTTATAACTTTAGGATTTAAT |
| | GCAGGTGACG |
| 2-PGK1t-R | ATCTTTCGAATTCACTGCCAAAACCTTGGGTCAATAGGATCTCCC |
| | ATGTCTCTACTGGT |
| 2-HcSDR-F | AAAGAAGCACCACCACCAGTAGAGACATGGGAGATCCTATTGA |
| | CCCAAGGTTTTGGCAG |
| 2-HcSDR-R | CTAATCTAAGTTTTAATTACAAAATGTCATCTAATGGTCAATCTC |
| | CATCC |
| 3-TEF1p-F | GATTGACCATTAGATGACATTTTGTAATTAAAACTTAGATTAGAT |
| | TGCTATGCTTT |
| 3-TEF1p-R | CTTACGATACCTGAGTATTCCCACAGTTAGTGATCCCCCACACAC |
| | CATAG |
| 3-LEU2-F | GAAGCTATGGTGTGTGGGGGATCACTAACTGTGGGAATACTCAG |
| | GTATCG |
| 3-LEU2-R | ATATAAAGGCGCCTGGCCGATTAAGCAAGGATTTTCTTAACTTC |
| | TTCGGC |
| 3-DN-F | CGAAGAAGTTAAGAAAATCCTTGCTTAATCGGCCAGGCGCCTTT |
| | ATATCA |
| 3-DN-R | GACTATAATATTATGCATATAGGATATACCAAAAATTCTCTCTG |

### 4. Identification of the Products Synthesized by Genetic Engineering Bacteria

The Saccharomyces cerevisiae engineering bacteria Cuol-04-1 is fermented and its fermentation products are collected, the method of fermentation and product collection is as follows: the chassis engineering bacteria Cuol-04-1 can produce
11-Carbonyl-cucurbita-5,23-diene-3β,20,25-triol (5),
11-Carbonyl-cucurbita-5,24-diene-3β,20,23-triol (5b),
11-Carbonyl-cucurbita-5,23-diene-20,25-diol-3β-one (5a),
11-Carbonyl-cucurbita-5,24-diene-20,23-diol-3β-one (5c) at a high level is activated in a solid screening culture plate, and the fermentation seed liquid is prepared in YPD liquid medium (30° C., 200 rpm, 16 hours); the fermentation is transferred to a 250 ml flask containing 50 ml YPD liquid medium (30° C., 200 rpm, 6 hours), and the expanded fermentation is transferred to a 3 L flask containing 1L YPD liquid medium at 30° C., 200 rpm/min. The fermentation product is obtained by shaking culture for 8 days.

Extraction conditions: The fermentation product is centrifuged at 8000 rpm for 10 min to collect the cells, the cells are soaked in 300 ml ethyl acetate for 30 min, ultrasonically extracted for 30 min shaken once every 10 min. The cells are collected by centrifugation at 8000 rpm for 10 min, and the supernatant is taken to detect the product.

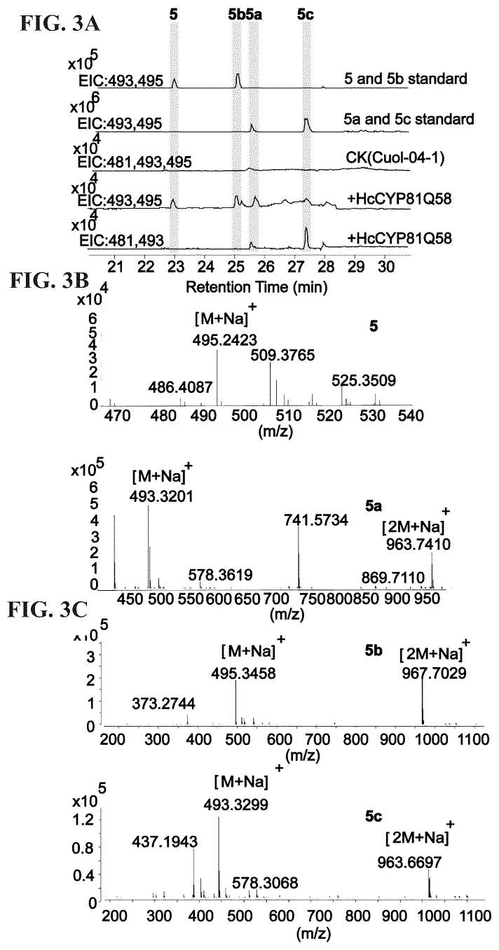

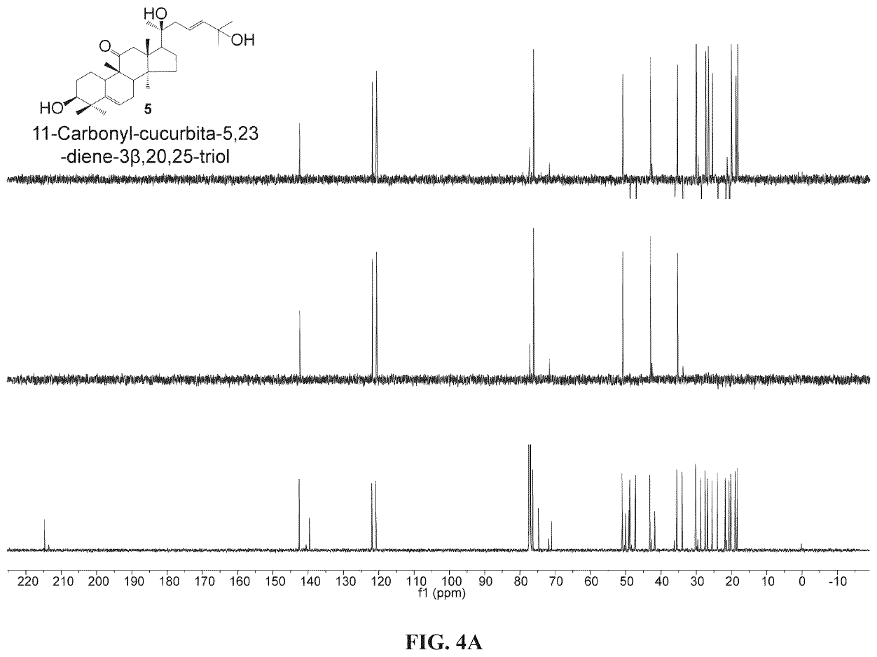

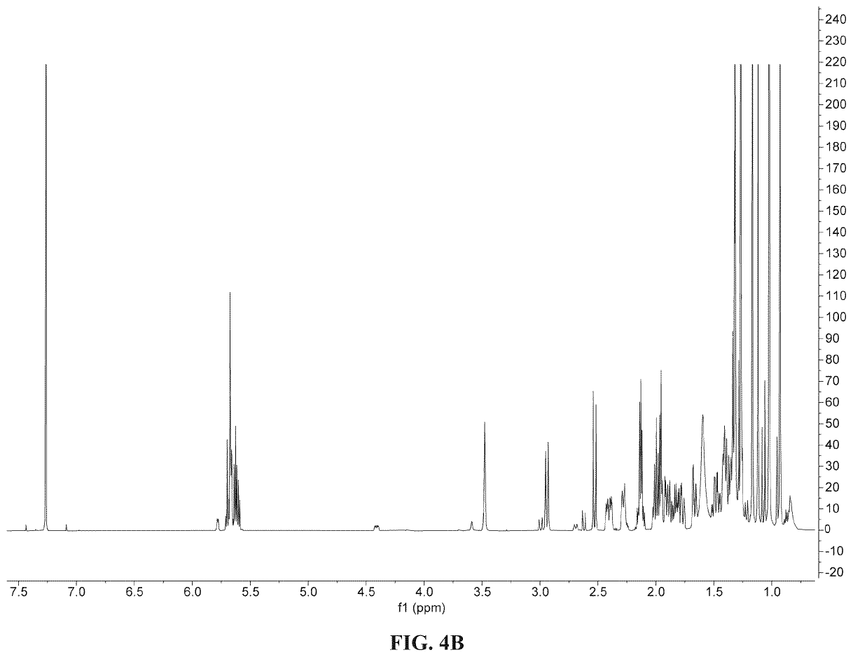

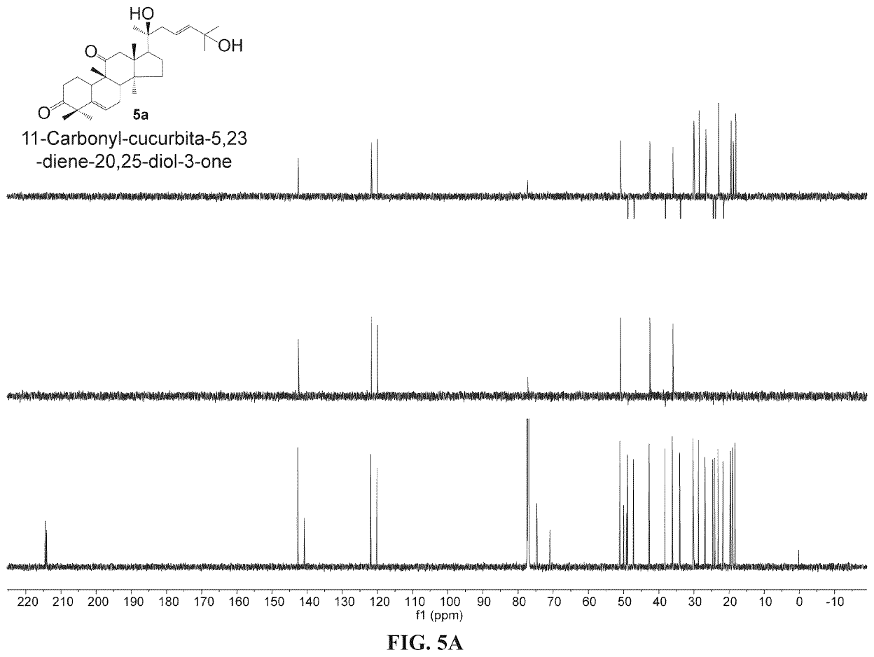

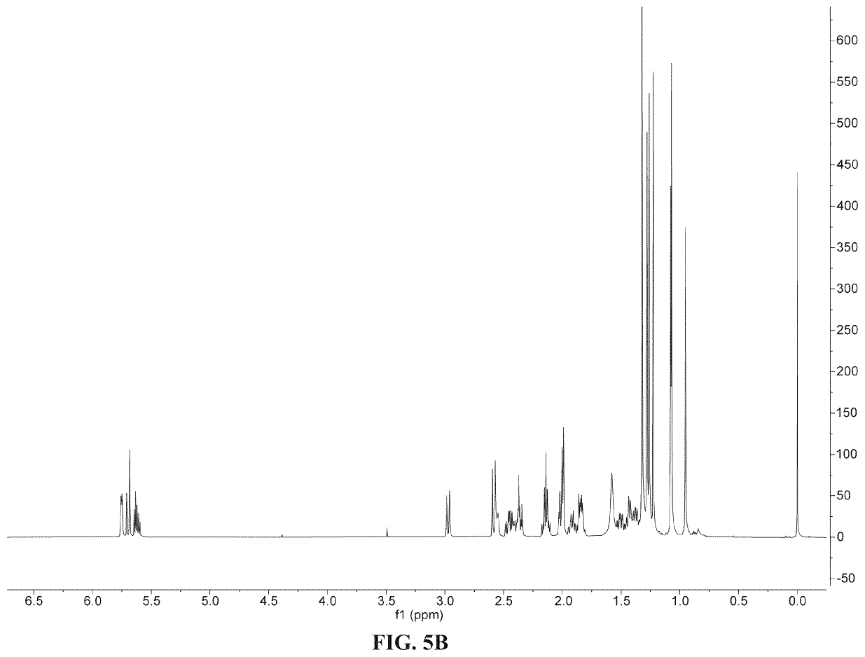

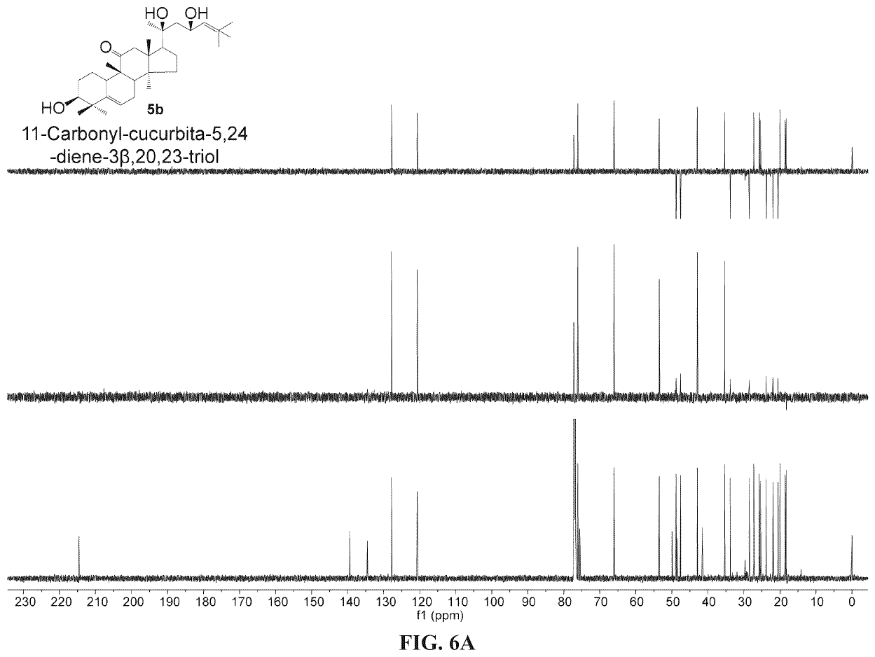

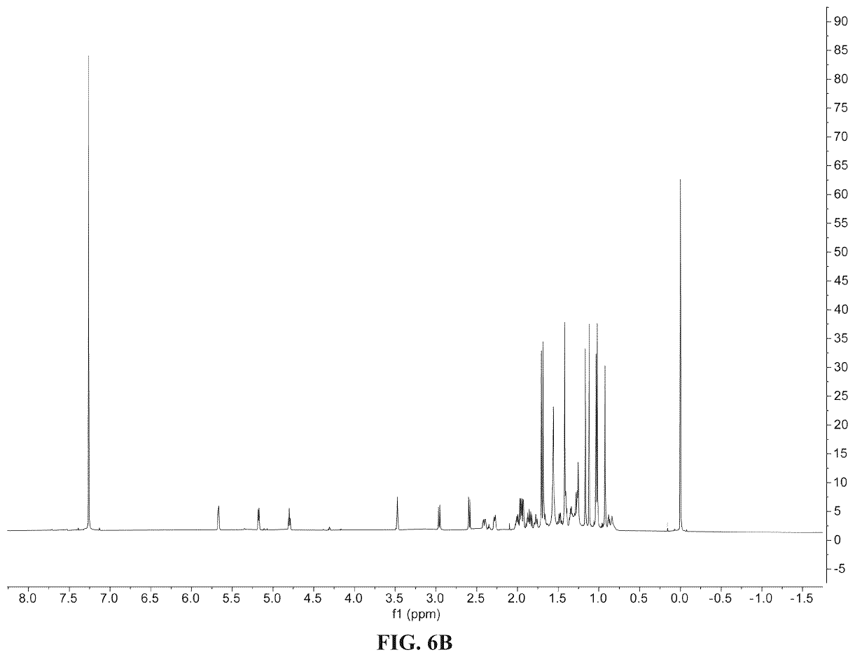

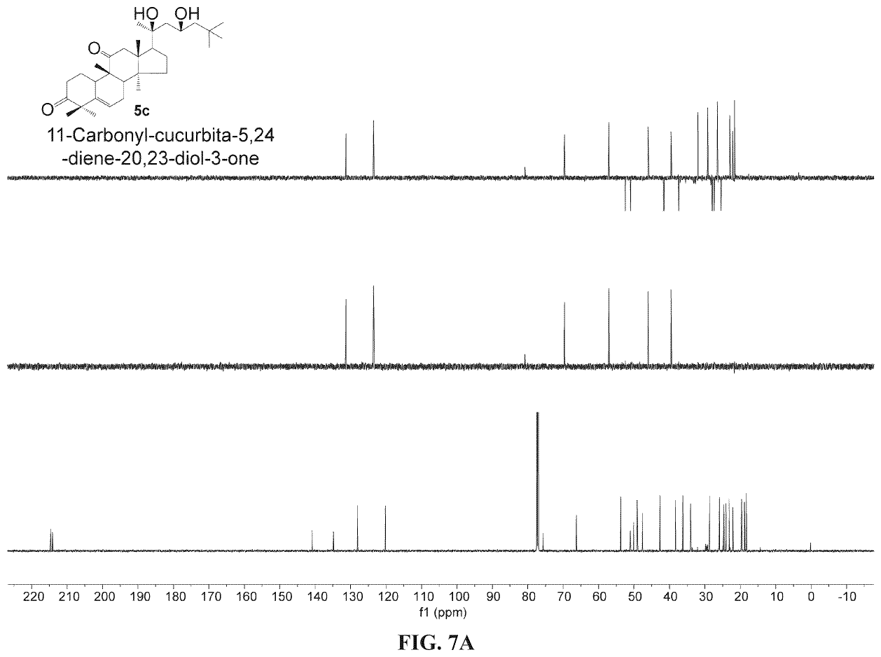

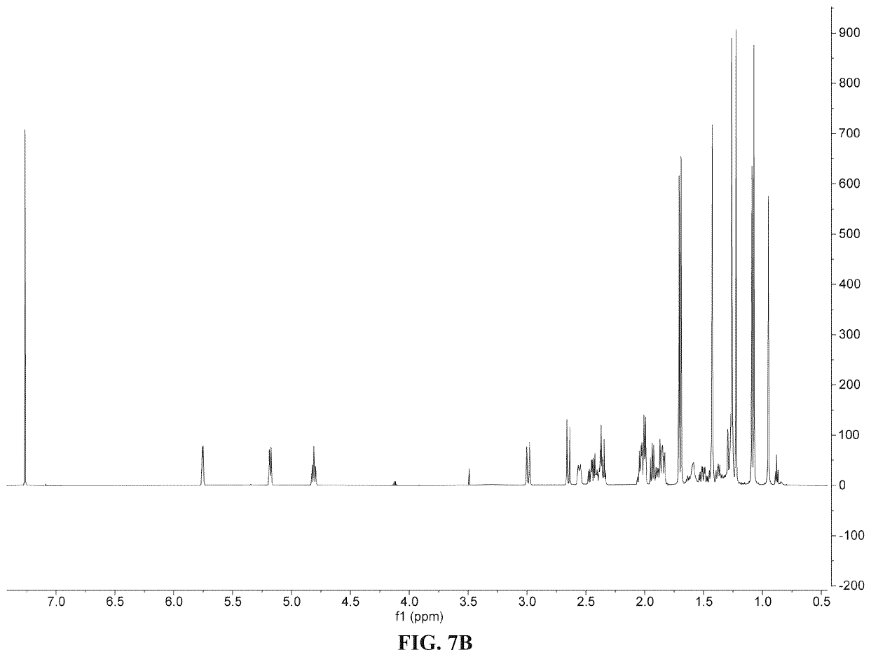

The fermentation products of Saccharomyces cerevisiae engineering bacteria Cuol-04-1 are identified by LC-Ms and NMR, the results are shown in Tables 2, 3, and FIGS. 3-7 (Among them, since the yield of 4 has a small yield, only LC-MS is detected, and it cannot be purified to be identified by NMR):

TABLE 2
Abbreviation and chemical nomenclature of compounds

| Abbreviation | Chemical nomenclature |
|---|---|
| 5 | 11-Carbonyl-cucurbita-5,24-diene-3β,20,23-triol |
| 5a | 11-Carbonyl-cucurbita-5,24-dicne-20,23-diol-3β-one |
| 5b | 11-Carbonyl-cucurbita-5,23-diene-3β,20,25-triol |
| 5c | 11-Carbonyl-cucurbita-5,23-diene-20,25-diol-3β-one |

TABLE 3
13C&1HNMR (800 MHz, solvent: CDC13) data of HcCYP81Q58 product
(J in Hz, δinppm) and Carbon numbering scheme and selected COSY, HMBC and NOESY 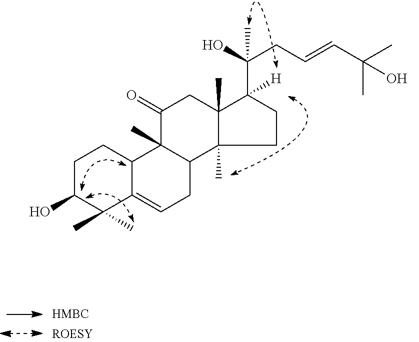
11-Carbonyl-cucurbita-5,23- diene-3β,20β,25-triol (5)

| Carbon | δ_{H} | δ_{C} |
|---|---|---|
| 1 | 1.40 (m), 1.48 (m) | 20.7 |
| 2 | 1.66(m), 1.78(m) | 28.7 |
| 3 | 3.48(t, J = 2.8 Hz) | 76.3 |
| 4 | / | 41.8 |
| 5 | / | 139.7 |
| 6 | 5.66 (m) | 120.8 |
| 7 | 1.94 (m),2.41 (m) | 24.0 |
| 8 | 1.96(m) | 43.2 |
| 9 | / | 48.8 |
| 10 | 2.27(m) | 35.5 |
| 11 | / | 214.7 |
| 12 | 2.53(d, J = 14.4 Hz),2.93 (d, J = 14.4 Hz) | 48.9 |
| 13 | / | 50.1 |
| 14 | / | 49.1 |
| 15 | 1.35(m), 1.40 (m) | 34.0 |
| 16 | 1.91(m), 1.99 (m) | 21.8 |
| 17 | 1.99 (m) | 51.0 |
| 18 | 0.93 (s, 3H) | 19.0 |
| 19 | 1.12 (s, 3H) | 20.3 |
| 20 | / | 74.7 |
| 21 | 1.27 (s, 3H) | 26.8 |
| 22 | 2.13 (m,2H) | 47.2 |
| 23 | 5.62 (m) | 122.0 |
| 24 | 5.68 (m) | 142.6 |
| 25 | / | 71.0 |
| 26 | 1.32 (s, 3H) | 30.0 |
| 27 | 1.32(s, 3H) | 30.1 |
| 28 | 1.17 (s, 3H) | 25.6 |
| 29 | 1.03 (s, 3H) | 27.5 |
| 30 | 1.02 (s, 3H) | 18.5 |

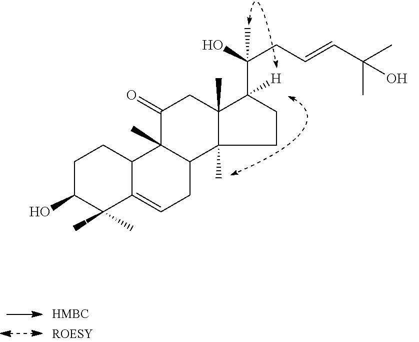

11-Carbonyl-cucurbita-5,23- diene-2β,25-diol-3-one (5a)

| Carbon | δH | δC |
|---|---|---|
| 1 | 1.50 (m), 1.86 (m) | 24.7 |
| 2 | 2.36 (m),2.44 (m) | 38.3 |
| 3 | / | 214.1 |
| 4 | / | 51.1 |
| 5 | / | 140.8 |
| 6 | 5.75 (dt, J = 5.2, 2.3 Hz) | 120.2 |
| 7 | 2.00 (m),2.40(m) | 24.1 |
| 8 | 1.99(m) | 42.8 |
| 9 | / | 49.0 |
| 10 | 2.55(m) | 36.2 |
| 11 | / | 214.5 |
| 12 | 2.58 (d, J = 14.4 Hz),2.95 (d, J = 14.5 Hz) | 49.0 |
| 13 | / | 49.1 |
| 14 | / | 50.0 |
| 15 | 1.37 (m), 1.42 (m) | 34.0 |
| 16 | 1.91 (m),2.01 (m) | 21.8 |
| 17 | 1.99 (m) | 51.1 |
| 18 | 0.95 (s, 3H) | 19.0 |
| 19 | 1.08 (s, 3H) | 19.6 |
| 20 | / | 74.7 |
| 21 | 1.28 (s, 3H) | 26.8 |
| 22 | 2.15 (m, 2H) | 47.3 |
| 23 | 5.62 (dt, J = 15.2, 7.3 Hz) | 121.9 |
| 24 | 5.69 (d, J = 15.5 Hz) | 142.7 |
| 25 | / | 71.0 |
| 26 | / | 30.2 |
| 27 | 1.32(s, 3H) | 30.3 |
| 28 | 1.26 (s, 3H) | 23.2 |
| 29 | 1.22 (s, 3H) | 28.7 |
| 30 | 1.07 (s, 3H) | 18.3 |

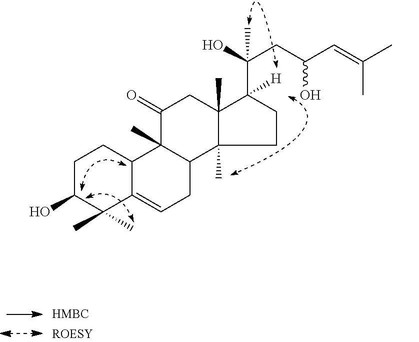

11-Carbonyl-cucurbita-5,24- diene-3β,20β,23-triol (5b)

| Carbon | δH | δC |
|---|---|---|
| 1 | 1.41 (m),1.47 (m) | 20.5 |
| 2 | 1.66 (m),1.77 (m) | 28.5 |
| 3 | 3.46 (d, J = 3.3 Hz) | 76.1 |
| 4 | / | 41.6 |
| 5 | / | 139.4 |
| 6 | 5.66 (dd, J = 5.1, 2.8 Hz) | 120.6 |
| 7 | 1.93 (m),2.41 (m) | 23.8 |
| 8 | 1.96(m) | 42.9 |
| 9 | / | 50.0 |
| 10 | 2.27 (d, J = 12.7 Hz) | 35.3 |
| 11 | / | 214.6 |
| 12 | 2.59 (d, J = 14.3 Hz),2.96 (d, J = 14.4 Hz) | 48.9 |
| 13 | / | 48.6 |
| 14 | / | 48.9 |
| 15 | 1.34 (m),1.39 (m) | 33.8 |
| 16 | 1.86 (m),1.99(m) | 21.9 |
| 17 | 1.93 (m) | 53.5 |
| 18 | 0.93 (s, 3H) | 18.6 |
| 19 | 1.12 (s, 3H) | 20.0 |
| 20 | / | 75.3 |
| 21 | 1.42 (s, 3H) | 25.8 |
| 22 | 1.26 (m), 1.83 (m) | 47.6 |
| 23 | 4.80 (ddd, J = 10.6, 8.4, 2.1 Hz) | 66.1 |
| 24 | 5.17 (d, J = 8.5 Hz) | 127.8 |
| 25 | / | 134.5 |
| 26 | 1.68 (s, 3H) | 18.2 |
| 27 | 1.70(s, 3H) | 25.7 |
| 28 | 1.16 (s, 3H) | 25.4 |
| 29 | 1.02 (s, 3H) | 27.3 |
| 30 | 1.03 (s, 3H) | 18.3 |

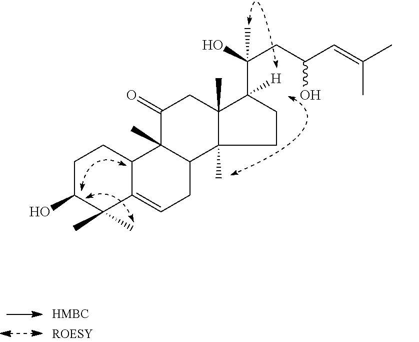

11-Carbonyl-cucurbita-5,24- diene-20β,23-diol-3-one (5c)

| Carbon | δH | δC |
|---|---|---|
| 1 | 1.50 (m), 1.84 (m) | 24.7 |
| 2 | 2.36 (m),2.44 (m) | 38.3 |
| 3 | / | 214.2 |
| 4 | / | 51.1 |
| 5 | / | 140.8 |
| 6 | 5.75 (dt, J = 5.3, 2.4 Hz) | 120.2 |
| 7 | 2.01 (m),2.38 (m) | 24.1 |
| 8 | 1.90(m) | 42.7 |
| 9 | / | 49.0 |
| 10 | 2.55(m) | 36.2 |
| 11 | / | 214.7 |
| 12 | 2.65 (d, J = 14.4 Hz),2.99 (d, J = 14.4 Hz) | 49.1 |
| 13 | / | 49.1 |
| 14 | / | 50.1 |
| 15 | 1.36 (m), 1.42 (m) | 34.1 |
| 16 | 1.93 (m),2.03 (m) | 22.1 |
| 17 | 1.92 (m) | 53.8 |
| 18 | 0.95 (s, 3H) | 18.8 |
| 19 | 1.08 (s, 3H) | 19.6 |
| 20 | / | 75.6 |
| 21 | 1.43 (s, 3H) | 25.9 |
| 22 | 1.27 (m),1.85 (m) | 47.7 |
| 23 | 4.82 (ddd, J = 10.6, 8.3, 2.1 Hz) | 66.3 |
| 24 | 5.18 (d, J = 8.4 Hz) | 128.0 |
| 25 | / | 134.9 |
| 26 | 1.69 (s, 3H) | 18.3 |
| 27 | 1.71(s, 3H) | 26.0 |
| 28 | 1.26 (s, 3H) | 23.2 |
| 29 | 1.23 (s, 3H) | 28.7 |
| 30 | 1.09 (s, 3H) | 18.4 |

It can be seen from the above table and FIGS. 3-7 that the Saccharomyces cerevisiae engineering bacteria Cuol-04-1 constructed by the invention has high yield, in which the yields of 11-Carbonyl-cucurbita-5,23-diene-3β, 20,25-triol and 11-Carbonyl-cucurbita-5,23-diene-20,25-diol-3β-one are 10.58 mg/L and 18.02 mg/L, respectively. 11-Carbonyl-Cucurbita-5,24-diene-3β, 20,23-triol and HcSDR34 catalyzes C3-ketone into 11-Carbonyl-cucurbita-5,24-diene-20,23-diol-3β-one with the yields of 12.58 mg/L and 16.02 mg/L, respectively.

Although the embodiment of the invention has been shown and described in the above content, it is understandable that the above embodiment is exemplary and cannot be understood as a restriction on the invention. Ordinary technicians in this field can change, modify, replace, and amend the above embodiments within the scope of the invention.

What is claimed is:

1. A HcCYP81Q58 gene of Hemsleya chinensis cytochrome oxidase, a sequence of the gene is shown in SEQ NO:1.

2. An application of the HcCYP81Q58 of Hemsleya chinensis cytochrome oxidase according to claim 1 in a preparation of cucurbitacin intermediates.

3. The application according to claim 2, wherein the cucurbitacin intermediates comprise:
11-Carbonyl-cucurbita-5,23-diene-3β,20,25-triol (5),
11-Carbonyl-cucurbita-5,24-diene-3β,20,23-triol (5b),
11-Carbonyl-cucurbita-5,23-diene-20,25-diol-3β-one (5a) and
11-Carbonyl-cucurbita-5,24-diene-20,23-diol-3β-one (5c).

4.-5. (canceled)

6. A method for constructing a HcCYP81Q58 transgenic Saccharomyces cerevisiae engineering bacteria of Hemsleya chinensis cytochrome oxidase, comprising the following steps:
controlling an expression of HcCYP81Q58 by using a constitutive promoter GPMp and a constitutive terminator IDP1t; controlling an expression of HcSDR34 by using a constitutive promoter TEF1p and a constitutive terminator PGK1t, and using LEU2 as a screening tag; constructing gene expression cassettes, the gene expression cassettes become downstream modules of a synthesis pathway of cucurbitacin intermediates; inserting gene expression cassettes into a γDNA site of a Saccharomyces cerevisiae by co-transformation, and carrying out an assembly by Saccharomyces cerevisiae homologous recombination ability to form Saccharomyces cerevisiae engineering bacteria containing a complete biosynthesis pathway of cucurbitacin intermediates.

7. The construction method according to claim 6, wherein the constitutive promoter is a combination of promoters GPMp and TEF1p.

8. The construction method according to claim 6, wherein the constitutive terminator is a combination of terminators IDP1t and PGK1t.

9. The construction method according to claim 6, wherein the gene expression cassettes also comprise a LEU2, the LEU2 is a screening gene.

10. The construction method according to claim 4, the cucurbitacin intermediates comprise:
11-Carbonyl-cucurbita-5,23-diene-3β, 20,25-triol (5),
11-Carbonyl-cucurbita-5, 24-diene-3β, 20, 23-triol (5b),
11-Carbonyl-cucurbita-5,23-diene-20,25-diol-3β-one (5a) and 11-Carbonyl-cucurbita-5,24-diene-20,23-diol-3β-one (5c).
